(19) United States
(12) Patent Application Publication
(10) Pub. No.: US 2022/0230766 A1
(43) Pub. Date: Jul. 21, 2022

(54) SYSTEM FOR PROVIDING A REMOTE THERAPY TRAINING PROGRAM

(71) Applicants: Amie Noelle Lowery-Luyties, Long Beach, CA (US); Michael Luyties, Long Beach, CA (US)

(72) Inventors: Amie Noelle Lowery-Luyties, Long Beach, CA (US); Michael Luyties, Long Beach, CA (US)

(21) Appl. No.: 17/568,798

(22) Filed: Jan. 5, 2022

Related U.S. Application Data
(60) Provisional application No. 63/138,853, filed on Jan. 19, 2021.

(51) Int. Cl. G16H 80/00; G16H 20/70; G06F 16/335

(52) U.S. Cl. CPC G16H 80/00; G06F 16/337; G16H 20/70

(57) ABSTRACT

A system for providing a psychotherapy training program, including a server configured to transmit information via a network to a trainer device and a therapist device. An application program is in operable communication with the server operating an example of an interactive display interface including an EMDR therapy training program including bilateral stimulation therapy and Brainspotting therapy. The bilateral stimulation therapy and Brainspotting therapy includes a lightbar continuously moving across an interactive display screen of the client device and the therapist device or one or more lights being placed on the client device by the therapist device, wherein the lightbar's movement is controlled by the therapist utilizing a lightbar controller and audio stimulation provided on the interactive display screen of the therapist device. The trainer device is operable to interact with the therapist device during a mock therapy session between at least two therapists and the at least one trainer.

### CROSS-REFERENCE TO RELATED APPLICATIONS

This application claims the benefit of U.S. Provisional Application No. 63/138,853 filed Jan. 19, 2021 and which is incorporated herein by reference.

### TECHNICAL FIELD

The embodiments generally relate to online training programs and more specifically relate to training programs for a web-based Eye Movement Desensitization and Reprocessing (EMDR) lightbar and audio bilateral stimulator to effect audio bilateral stimulation (BLS) via the Internet or world-wide web, in reliance upon the web-based lightbar and audio bilateral stimulator.

### BACKGROUND

EMDR is a method of psychological therapy for the treatment of a range of psychological disorders and mental health problems including trauma, anxiety, Post-traumatic Stress Disorder (PTSD), among others. During EMDR therapy, the therapist aids the client in processing distressing experiences and guides the client through a series of BLS's, involving side-to-side sensory stimulation. Often, a light is moved back and forth across a screen, a sound is switched between speakers on either side of the client, a vibration is moved back and forth between vibrating devices, or a combination thereof. Techniques similar to EMDR, including Brainspotting, may also include vertical stimulation and lateral stimulation while focusing on one or more fixed points.

Online therapies are often provided on a collaborative platform between the therapist and the client and require the therapist to control the functionalities of the system using the interactive display on their computing device. In the current arts, therapists are often left to self-train using their computerized therapy programs.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a system for providing a psychotherapy training program, including a server configured to transmit information via a network to a trainer device and a therapist device. An application program is in operable communication with the server, the application program to operate an example of an interactive display interface including an EMDR therapy training program including bilateral stimulation therapy and Brainspotting therapy. The bilateral stimulation therapy and Brainspotting therapy is comprised of a lightbar continuously moving across an interactive display screen of the client device and the therapist device, and wherein the lightbar's movement is controlled by the therapist utilizing a lightbar controller provided on the interactive display screen of the therapist device. The trainer device is operable to interact with the therapist device during a mock therapy session between one or more therapists and one or more trainers.

The system allows the trainers and therapists to cooperatively engage in utilizing the EMDR therapy system in a training environment such that the therapist can learn the various functionalities of the system with the oversight of a trainer. This reduces the time needed to learn the functionalities of the therapy system while ensuring the therapy system is utilized properly during a therapy session with a client.

Attendees may be separated into groups during the mock therapy sessions to allow for therapists to perform a mock therapy session on other therapists. The trainer(s) may enter and exit the mock therapy sessions to spectate and provide any necessary guidance or instruction. The trainer(s) may toggle between entering and exiting the smaller mock therapy sessions and exiting and entering the large group training session.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a block diagram of the application system, according to some embodiments;

FIG. 3 illustrates an example of an interactive display interface wherein the trainer establishes a training session and selects attendees (i.e., therapists) who will attend the training program, according to some embodiments;

FIG. 4 illustrates an example of an interactive display interface wherein the trainer and therapists are interacting on a video conferencing system prior to engaging in the mock therapy session, according to some embodiments;

FIG. 5 illustrates an example of an interactive display interface including the training session controller, according to some embodiments;

FIG. 6 illustrates an example of an interactive display interface including the stream setup controls, according to some embodiments;

FIG. 7 illustrates an example of an interactive display interface showing the video stream and stream setup controls, according to some embodiments; and

FIG. 8 illustrates an example of an interactive display interface showing the video stream and training lobby controls, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to systems for providing an online training system for EMDR-based or Brainspotting therapies. One skilled in the arts will readily understand that the training system may be used for a variety of online therapies disciplines known in the arts. The system allows the trainers and therapists to cooperatively engage with one another utilizing the EMDR therapy system or Brainspotting therapy system in a training environment such that the therapist can learn the various functionalities of the system with the oversight and interactive guidance of a trainer. This reduces the time needed to learn the functionalities of the therapy system while ensuring the therapy system is utilized properly during a therapy session with a client.

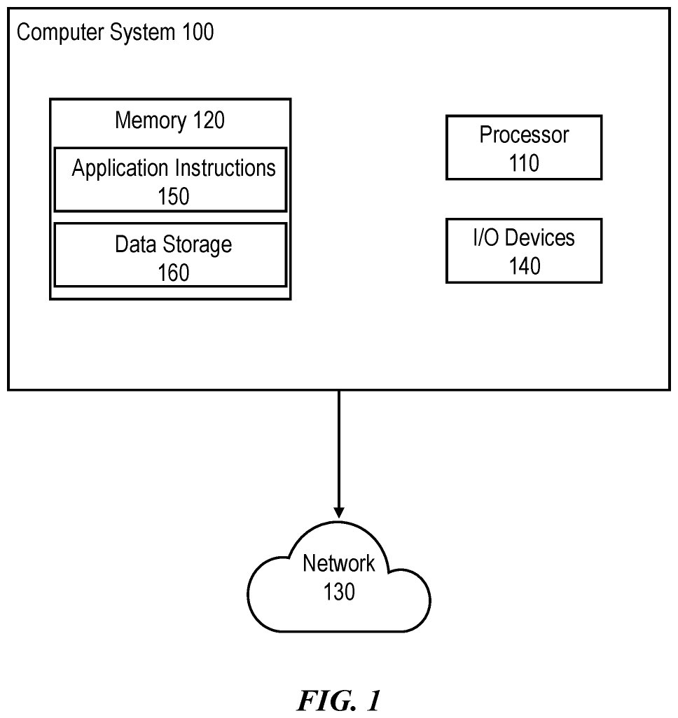

FIG. 1 illustrates a computer system 100, which may be utilized to execute the processes described herein. The computer system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. A computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages or scripting languages (e.g., C, C++, C #, JAVA®, JAVASCRIPT®, PERL®, etc.).

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

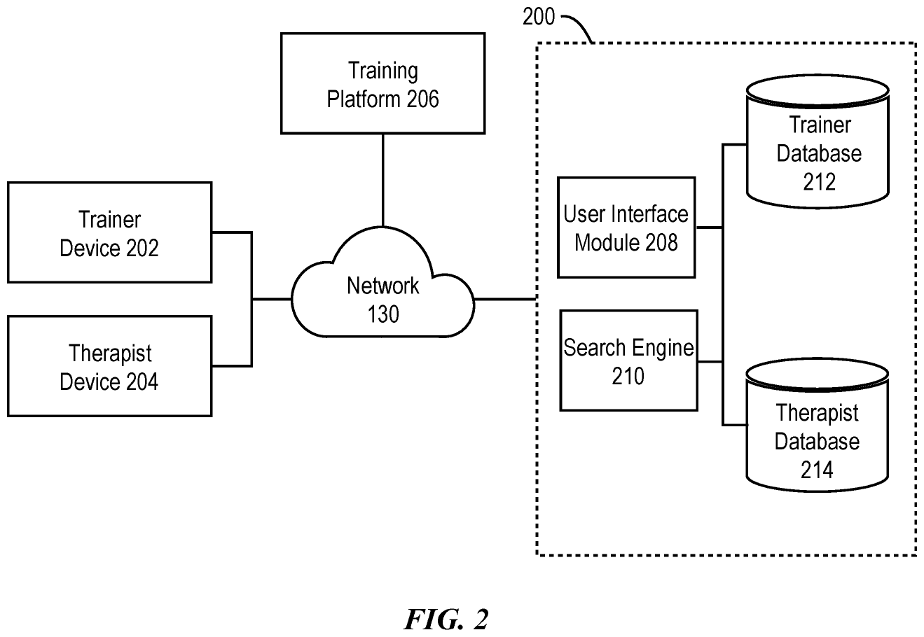

FIG. 2 illustrates a block diagram of the application system 200 in operable communication with the trainer device 202 and the therapist device 204 via a network 130. The trainer utilizes their trainer device 202 (e.g., a desktop computer, laptop, tablet, smartphone, etc.) to interact with the therapist using a therapist device 204 and engage in the training platform 206. The training platform 206 provides the functionalities of an online EMDR-based therapy or Brainspotting therapy platform which can be used by therapists to provide remote therapy to a client. The training platform 206 allows for the therapist to learn and practice therapy techniques with trainers or other therapists. The application system 200 includes a user interface module 208 and search engine 210 to allow the user to search and display information stored in a trainer database 212 and therapist database 214. The trainer database 212, for example, may store trainer information such as trainer login credentials, trainer permissions, trainer personal information, etc. The therapist database 214, for example, may store therapist information such as therapist login credentials, therapist personal information, therapist permissions, etc.

The trainer device 202 and the therapist device 204 can be utilized to interact with one another via the remote therapy training program. Each device 202,204 may be utilized by the respective user to exchange information related to undergoing and providing training for the remote therapy training system. Trainers may utilize the system to create a training session and create groups within the training session.

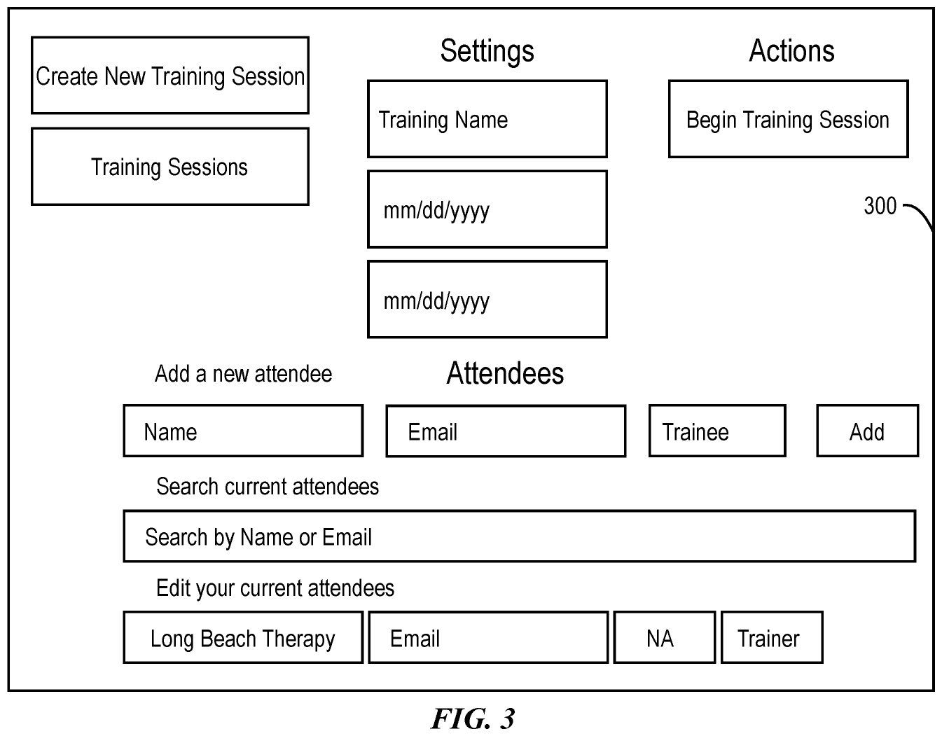

FIG. 3 illustrates an example of an interactive display interface 300 wherein a plurality of training settings are input by the user. The training settings may include the training name, day and time of the training session, and the attendees (therapists or trainers) who will attend the training session at the user indicated day and time of the training session. The user may also select to begin a training session to initiate the training session which is accessible by the trainer(s) and therapists. The training session may be accessed via a web link transmitted via email or similar form of communication. Attendees may be added or removed before, during, or after the training session.

In some embodiments, the attendees may be separated into groups which may be separated from the training session. Groups may allow attendees to interact in a real-time mock therapy session with other attendees. For example, one or more therapists may undergo a mock therapy session with other therapists or with trainers. In some embodiments, the trainers may be able to readily enter and exit the groups to provide guidance and instruction to the therapists or to spectate the mock therapy session.

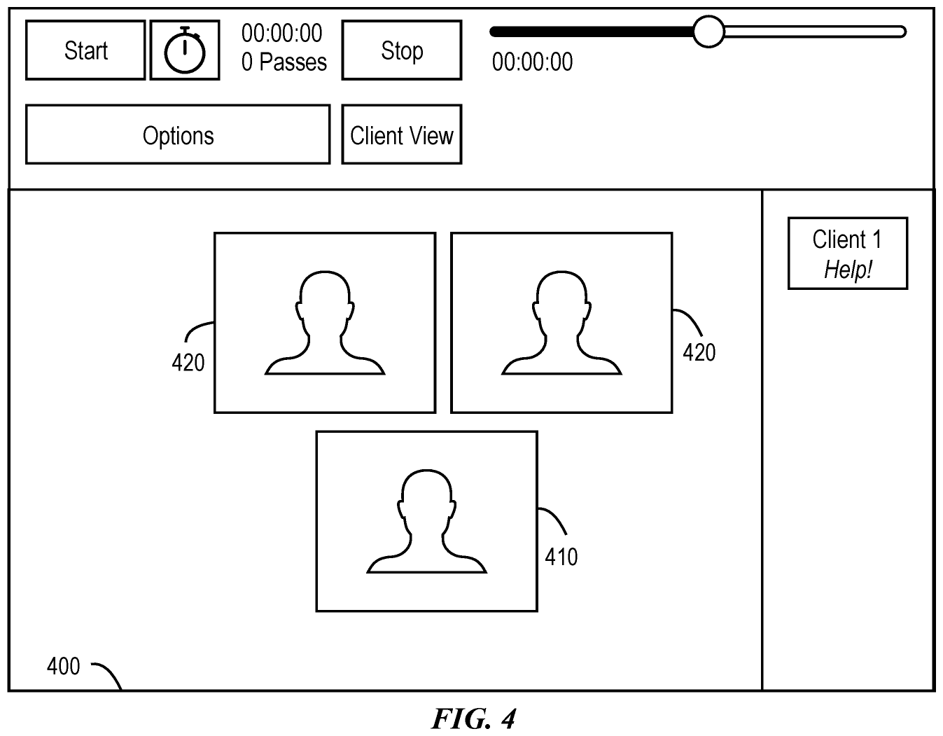

FIG. 4 illustrates an interactive display training interface 400 wherein the trainer 410 and attendees 420 (i.e., the therapists) are interacting via a video conference. The interactive display training interface may be utilized for the entire group of attendees or the groups during a mock therapy exercise. Controls are provided including a “START” button to begin the therapy session or to begin the live broadcast of the training program. A “STOP” button allows the user to selectively stop the training session or mock therapy session. The session duration may be timed and saved for reference by the trainer or the therapists while undergoing the training or the mock therapy session.

During the mock therapy session, the remote EMDR or Brainspotting tools are made available to the therapists. This includes the controls for the lightbar as well as audio capabilities to allow for communications between the therapist and the trainer.

In some embodiments, a chat box is available to allow attendees of the training session to communication with one another. For example, the chat box may be utilized to ask questions, get the attention of the trainer, or to provide communications if a user does not have a microphone and audio capabilities.

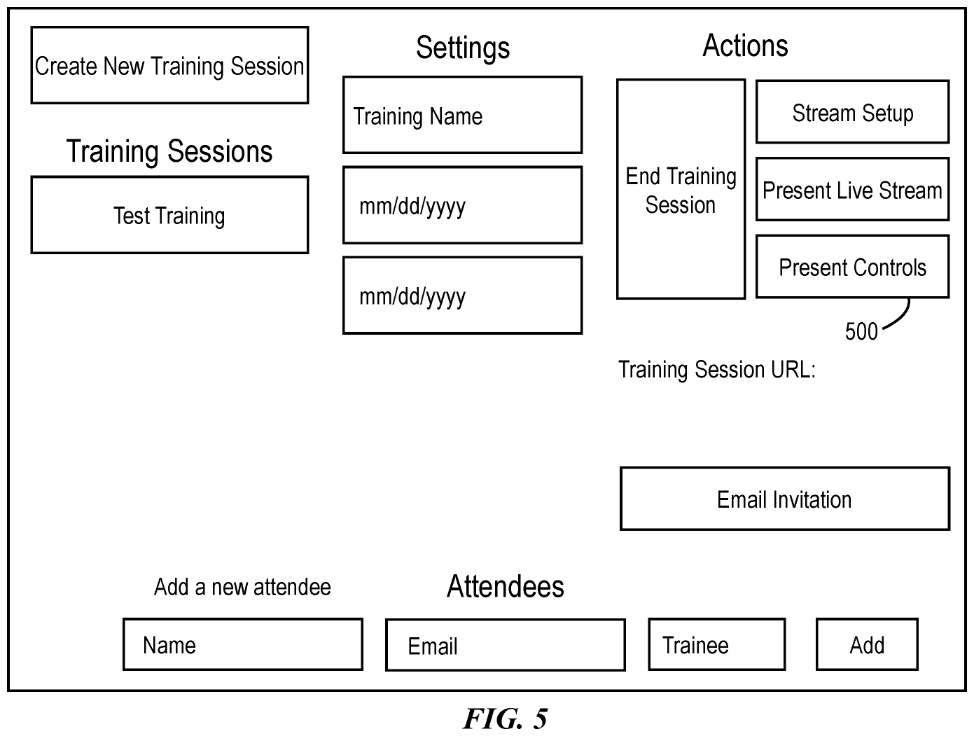

FIG. 5 illustrates an example of an interactive display interface including the training session controls 500 which can be used by the trainer during a mock therapy training session. The controls include the functions to start a training session, end a training session, setup a stream (e.g., using a web-based streaming platform), present a live stream, and present controls. The system may also generate a URL which can be transmitted to attendees, thus allowing the attendees to access the training session remotely.

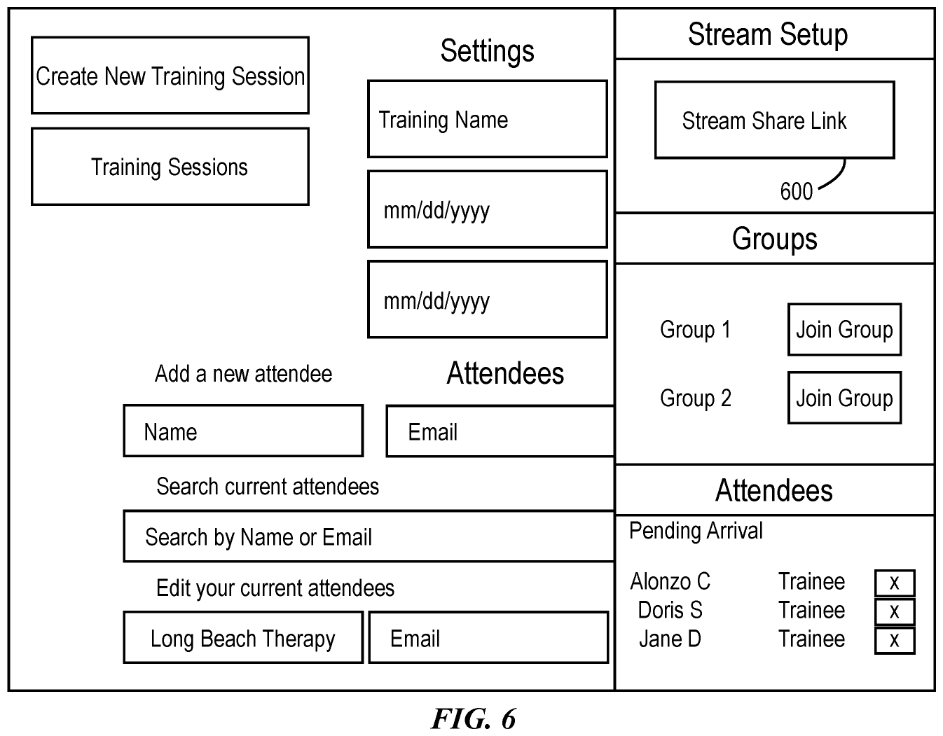

FIG. 6 illustrates the example of an interactive display interface having stream setup controls 600 which allow the trainer to move in an out of groups within the training session. For example, the trainer may separate the attendees into separate groups (such as Group 1 and Group 2) which can be used for mock therapy sessions between attendees. The trainer can also see a listing of attendees. The listing allows the trainer to add, remove, or otherwise interact with the attendees. The listing of attendees may also indicate the status for each attendee (e.g., currently in a training session, not yet joined, etc.)

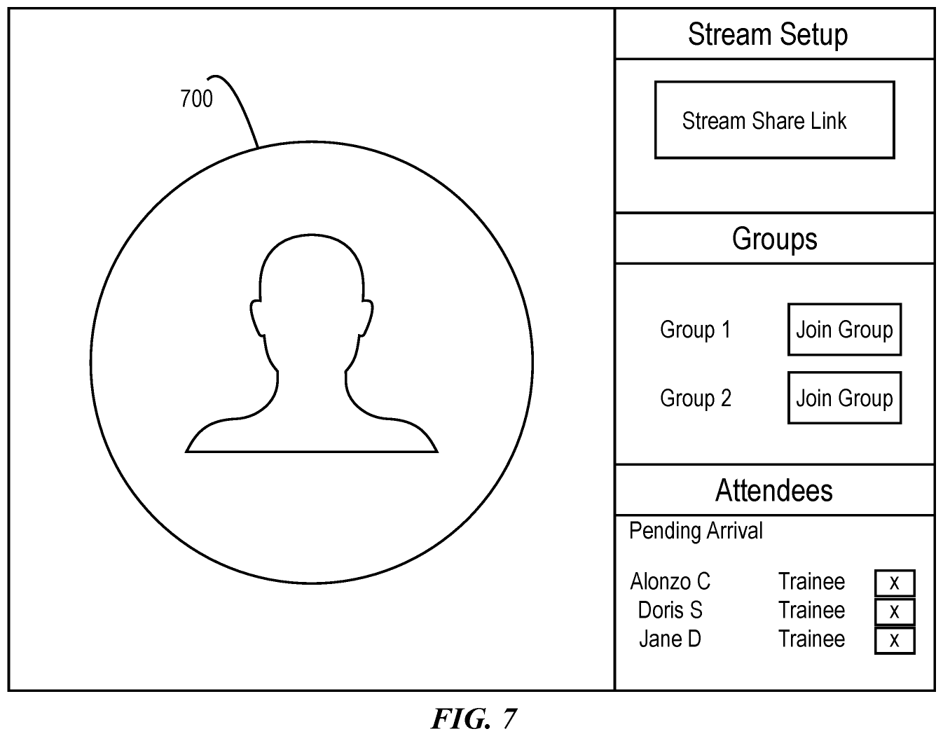

FIG. 7 illustrates an example of an interactive display interface showing the video stream 700 and stream setup controls. The video stream 700 is displayed once the stream is setup and started by the trainer. The video stream may include one or more trainers or attendees.

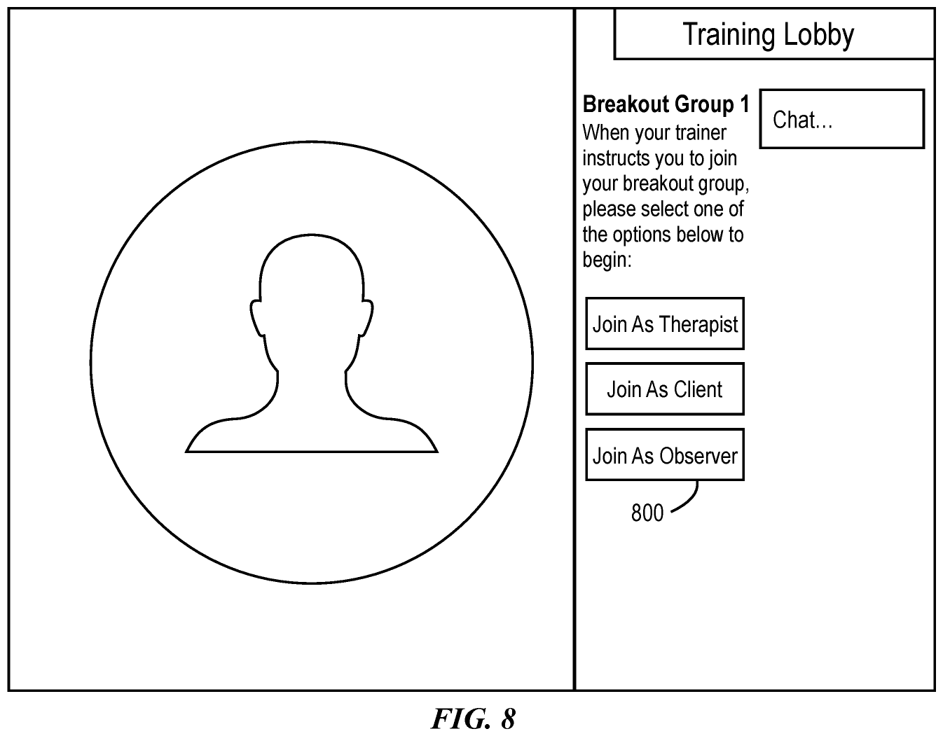

FIG. 8 illustrates an example of an interactive display interface showing the video stream and training lobby controls 800 including a plurality of selectable tabs comprising a join as therapist tab, a join as client tab, and a join as observer tab. The lobby controls interface 800 also provides a chat feature to permit users to communicate between one another.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, a system for providing a psychotherapy training program may include a server configured to transmit information via a network to a trainer device and a therapist device; an application program in operable communication with the server, the application programmed to operate an example of an interactive display interface including an EMDR therapy training program including bilateral stimulation therapy, wherein the bilateral stimulation therapy is included of a lightbar continuously moving across an interactive display screen of the client device and the therapist device, and wherein the lightbar's movement is controlled by the therapist utilizing a lightbar controller provided on the interactive display screen of the therapist device; and wherein the trainer device is operable to interact with the therapist device during a mock therapy program between at least one therapist and the at least one trainer.

Variation 2 may include a system as in variation 1, wherein the mock therapy session includes audio stimulation.

Variation 3 may include a system as in any of variations 1 through 2, wherein the lightbar is an eye movement desensitization and reprocessing lightbar and the audio stimulation is an audio bilateral stimulator constructed and arranged to effect audio bilateral stimulation.

Variation 4 may include a system as in any of variations 1 through 3, further including a memory to store attendee data.

Variation 5 may include a system as in any of variations 1 through 4, wherein the trainer and therapist connect to a website managed by the server via the Internet.

Variation 6 may include a system as in any of variations 1 through 5, wherein the step of using a computer server includes managing and preparing a database of therapists and a database of trainers.

Variation 7 may include a system as in any of variations 1 through 6, wherein the system further includes at least one of a trainer database, therapist database, user interface module, or search engine.

Variation 8 may include a system as in any of variations 1 through 7, wherein the trainer database is configured to allow a user to search information stored in a trainer database and therapist database and wherein the system is configured to display search information on a display of a device.

Variation 9 may include a system as in any of variations 1 through 8, wherein the therapist database is configured to store therapist information including at least one of therapist login credentials, therapist personal information, or therapist permissions.

Variation 10 may include a system as in any of variations 1 through 9, wherein the user interface module and search engine are configured to cooperatively allow a user to search and display information on a display on at least one of a trainer device or a therapist device.

According to variation 11, a system for providing a psychotherapy training program may include a server configured to transmit information via a network to a trainer device and a therapist device; and an application program in operable communication with the server. The application program may be configured to operate an example of an interactive display interface including an EMIDR therapy training program including bilateral stimulation therapy, wherein the bilateral stimulation therapy is included of a lightbar continuously moving across an interactive display screen of the client device and the therapist device, and wherein the lightbar's movement and audio stimulation is controlled by the therapist utilizing a lightbar controller provided on the interactive display screen of the therapist device; the trainer device operable to interact with a therapist device during a mock therapy program between at least one therapist and the at least one trainer; and wherein the at least one therapist and the at least one trainer are separated into one or more groups including at least two therapists, wherein the at least one trainer interacts with each of the one or more groups within the mock therapy program.

Variation 12 may include a system as in variation 11, wherein the system further includes at least one of a trainer database, therapist database, user interface module, or search engine.

Variation 13 may include a system as in any of variations 11 through 12, wherein the trainer database is configured to allow a user to search information stored in a trainer database and therapist database and wherein the system is configured to display search information on a display of a device.

Variation 14 may include a system as in any of variations 10 through 13, wherein the therapist database is configured to store therapist information including at least one of therapist login credentials, therapist personal information, or therapist permissions.

Variation 15 may include a system as in any of variations 10 through 14, wherein the user interface module and search engine are configured to cooperatively allow a user to search and display information on a display on at least one of a trainer device or a therapist device.

Variation 16 may include a system as in any of variations 10 through 15, wherein EMDR therapy training program is controlled by the therapist utilizing a lightbar controller provided on the interactive display screen of the therapist device.

According to variation 17, a system for providing a psychotherapy training program may include at least one trainer device; at least one therapist device in operable communication with the at least one trainer device via a network; a server configured to transmit information via the network to the at least one trainer device and the at least one therapist device, the server including a memory that stores computer-executable components and a processor that executes the computer-executable components stored in the memory. The computer-executable components may include an application in operable communication with the server, the application including at least one of a trainer database, therapist database, user interface module, or search engine; the application programmed to operate an example of an interactive display interface including at least one of an EMDR therapy training program including bilateral stimulation therapy or mock therapy program between at least one therapist and the at least one trainer.

Variation 18 may include a system as in variation 17, wherein the EMDR therapy training program includes an eye movement desensitization and reprocessing lightbar and an audio bilateral stimulator constructed and arranged to effect audio bilateral stimulation.

Variation 19 may include a system as in any of variations 17 through 18, wherein the bilateral stimulation therapy is included of a lightbar continuously moving across an interactive display screen of the client device and the therapist device

Variation 20 may include a system as in any of variations 17 through 19, wherein the lightbar's movement is controlled by the therapist utilizing a lightbar controller provided on the interactive display screen of the therapist device

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for providing a psychotherapy training program, the system comprising:
a server configured to transmit information via a network to a trainer device and a therapist device;
an application program in operable communication with the server, the application programmed to operate an example of an interactive display interface including an EMDR therapy training program including bilateral stimulation therapy, wherein the bilateral stimulation therapy is comprised of a lightbar continuously moving across an interactive display screen of the client device and the therapist device, and wherein the lightbar's movement is controlled by the therapist utilizing a lightbar controller provided on the interactive display screen of the therapist device; and
wherein the trainer device is operable to interact with the therapist device during a mock therapy program between at least one therapist and the at least one trainer.

2. The system of claim 1, wherein the mock therapy session comprises audio stimulation.

3. The system of claim 2, wherein the lightbar is an eye movement desensitization and reprocessing lightbar and the audio stimulation is an audio bilateral stimulator constructed and arranged to effect audio bilateral stimulation.

4. The system of claim 1, further comprising a memory to store attendee data.

5. The system of claim 1, wherein the trainer and therapist connect to a website managed by the server via the Internet.

6. The system of claim 1, wherein the step of using a computer server comprises managing and preparing a database of therapists and a database of trainers.

7. The system of claim 1, wherein the system further comprises at least one of a trainer database, therapist database, user interface module, or search engine.

8. The system of claim 7, wherein the trainer database is configured to allow a user to search information stored in a trainer database and therapist database and wherein the system is configured to display search information on a display of a device.

9. The system of claim 7, wherein the therapist database is configured to store therapist information including at least one of therapist login credentials, therapist personal information, or therapist permissions.

10. The system of claim 7, wherein the user interface module and search engine are configured to cooperatively allow a user to search and display information on a display on at least one of a trainer device or a therapist device.

11. A system for providing a psychotherapy training program, the system comprising:
a server configured to transmit information via a network to a trainer device and a therapist device;
an application program in operable communication with the server, the application program to operate an example of an interactive display interface including an EMDR therapy training program including bilateral stimulation therapy, wherein the bilateral stimulation therapy is comprised of a lightbar continuously moving across an interactive display screen of the client device and the therapist device, and wherein the lightbar's movement and audio stimulation is controlled by the therapist utilizing a lightbar controller provided on the interactive display screen of the therapist device;
the trainer device operable to interact with a therapist device during a mock therapy program between at least one therapist and the at least one trainer; and
wherein the at least one therapist and the at least one trainer are separated into one or more groups comprising at least two therapists, wherein the at least one trainer interacts with each of the one or more groups within the mock therapy program.

12. The system of claim 11, wherein the system further comprises at least one of a trainer database, therapist database, user interface module, or search engine.

13. The system of claim 12, wherein the trainer database is configured to allow a user to search information stored in a trainer database and therapist database and wherein the system is configured to display search information on a display of a device.

14. The system of claim 12, wherein the therapist database is configured to store therapist information including at least one of therapist login credentials, therapist personal information, or therapist permissions.

15. The system of claim 12, wherein the user interface module and search engine are configured to cooperatively allow a user to search and display information on a display on at least one of a trainer device or a therapist device.

16. The system of claim 11, wherein EMDR therapy training program is controlled by the therapist utilizing a lightbar controller provided on the interactive display screen of the therapist device.

17. A system for providing a psychotherapy training program, the system comprising:
at least one trainer device;
at least one therapist device in operable communication with the at least one trainer device via a network;
a server configured to transmit information via the network to the at least one trainer device and the at least one therapist device, the server comprising a memory that stores computer-executable components and a processor that executes the computer-executable components stored in the memory, wherein the computer-executable components comprise:
an application in operable communication with the server, the application comprising at least one of a trainer database, therapist database, user interface module, or search engine;
the application programmed to operate an example of an interactive display interface including at least one of an EMDR therapy training program including bilateral stimulation therapy or mock therapy program between at least one therapist and the at least one trainer.

18. The system of claim 17, wherein the EMDR therapy training program comprises an eye movement desensitization and reprocessing lightbar and an audio bilateral stimulator constructed and arranged to effect audio bilateral stimulation.

19. The system of claim 17, wherein the bilateral stimulation therapy is comprised of a lightbar continuously moving across an interactive display screen of the client device and the therapist device

20. The system of claim 17, wherein the lightbar's movement is controlled by the therapist utilizing a lightbar controller provided on the interactive display screen of the therapist device
